(19) United States
(12) Patent Application Publication
(10) Pub. No.: US 2017/0081056 A1
(43) Pub. Date: Mar. 23, 2017

(54) METHOD OF SEALING A CONTAINER COMPRISING AT LEAST ONE PLUG, PARTICULARLY A CARPULE, INSERTION MEANS AND ASSOCIATED SEALING LINE

(71) Applicant: DISPOSABLE-LAB, MARTILLAC (FR)

(72) Inventors: Jean-Pascal ZAMBAUX, BOULIAC (FR); Bernard PAUCHDET, SAINT CAPRAISE DE LALINDE (FR)

(21) Appl. No.: 15/272,174

(22) Filed: Sep. 21, 2016

(30) Foreign Application Priority Data
Sep. 21, 2015 (FR) 15 58862

(51) Int. Cl. B65B 7/16; B65B 7/28; A61M 5/30; A61J 1/06; A61J 1/20; A61M 19/00

(52) U.S. Cl. CPC B65B 7/161; A61J 1/20; A61M 19/00; A61M 5/30; A61J 1/062; B65B 7/2821

(57) ABSTRACT

Disclosed is a method of sealing a container with a liquid, the container including a body with an opening at at least one end and including at least one sealing plug, designed to seal the opening after filling, the filling defining a free surface S for filling, the filling having allowed a head space to remain in the container. The method includes: introduction of at least one rod parallel to an inside wall of the body of the container; mechanical insertion of the plug in the body, along the at least one insertion rod, generating at least one channel C, the air of the head space being evacuated between the plug and the rod; and withdrawal of the at least one rod while leaving the plug in place at the level of insertion of the preceding step. Also disclosed are the associated device and the industrial production line.

### BACKGROUND OF THE INVENTION

Field Of The Invention

This invention relates to a method of sealing a container comprising at least one plug, particularly a carpule.

The invention also covers the insertion means associated with its different variants.

Finally, the invention covers a line for sealing containers, in an industrial way.

For the remainder of the description, the choice employed for illustration in a nonlimiting way is an application having a carpule for the medical field.

The description would apply just as well to containers of which one end is sealed and which must be filled and sealed at the second end by a plug that enters into said container, while producing a minimum of air, such as a syringe body.

Description Of The Related Art

Actually, in the medical field, intradermal syringe injection devices are highly used, but many patients can exhibit a phobia of needles and of the act caused by the needle. Thus, children can be sensitive to it but adults as well, especially when it involves a repetitive treatment such as treatments for allergies or for daily doses in the case of long-term treatments.

Also, to deal with the situation, there are needle-free injection means, particularly means distributed under the trademark Zeneo registered by the CrossJect Company.

Such means operate by propelling the liquid contained in a carpule by means of a very high localized pressurization so as to cause the liquid contained in said carpule to penetrate through the skin. An injection head ensures the diffusion of the liquid into numerous streams of very small diameters.

Therefore, there is no longer a needle, and it is enough to position an applicator on the skin and to initiate by simple pressure the use of the means for placing under very high pressure, which triggers the injection of the liquid by jetting at very high speed through the skin.

It is understood that if the liquid must be placed under very high pressure, almost instantaneously the carpule containing said liquid must be free of air or at least the volume of air must be reduced as much as possible so as to absorb as little as possible the pressure wave exerted by the means for placing under very high pressure.

The more complete the quasi-instantaneous pressure transmitted to the liquid, the more effective and high-quality the penetration will be.

Now, the present-day filling can be greatly improved.

### SUMMARY OF THE INVENTION

A carpule consists of a container that takes the shape of a tube, generally made of glass, of great thickness to withstand the pressure, with a peripheral lip at the ends for its hold and positioning.

This tube-shaped container is sealed at a proximal first end by a first plug, fully introduced into the tube, by one end. This plug is provided with means for fluidtightness with the wall of said tube. This plug, thus positioned, remains nevertheless susceptible of being moved under the high pressure that will be exerted during its use so as to free the liquid contained in said tube through this proximal end that is equipped with a diffusion head. This diffusion head has as its object to split the diffused volume into a considerable number of jets of very small diameters.

During the manufacture, the tube-shaped container, sealed by this proximal plug and designed to carry a diffusion head, must then be filled with the liquid to be dispensed through its distal other end. This distal end is then sealed by a distal plug, pending its use.

The distal plug must also enter into the tube completely, on the one hand, because the dimensions of the tube-shaped container are standard and because the volume of liquid to be dispensed can be variable, and, on the other hand, because the second plug is going to receive and transmit the very high pressure generated. This second plug will then behave as a plunger to transmit this very high pressure by the action of the means for placing under very high pressure.

In addition, the carpules are used as cartridges in other fields such as the dental field, for example for the administration of anesthesia products. In this case, this administration necessarily resorts to a syringe into which the carpule, also called a cartridge in this professional environment, is introduced directly. Here also, the volume of air must be limited to transmit the pressure exerted on the plunger directly to the anesthetizing fluid, without a buffer volume of air that would absorb a portion of the force or at least with a minimum volume of air that would render the damping capacity negligible.

The anesthetizing fluids most often exhibit a considerable viscosity necessitating a high pressure from the medical practitioner to ensure a good penetration of the anesthetizing product into the gum.

The carpules are also used in the veterinary field where the problem of filling the cartridges and other carpules with a minimum of air also occurs.

A known solution is to fill and seal a carpule with a plug by plugging the carpule by its proximal end, in filling the carpule, in putting the carpule under vacuum, in introducing the distal plug when the carpule is under vacuum, and then in breaking the vacuum. The distal plug is then moved in the tube that constitutes the carpule.

This method exhibits a first problem linked to the control of the movement in translation of the first plug.

The reproducibility is challenging and, especially in the case of an error or of an incomplete movement, there is no possibility of “completing” the movement or of recommencing or even of recreating the vacuum. The contents of the carpule are then lost, which can be very detrimental when the product has a high cost.

Generally, the distal plug consists of a plug body comprising at least two annular lips, generally three, located one above the other along the longitudinal axis of the carpule and spaced apart from one another.

These lips form the fluidtightness of the distal plug and constitute the same number of barriers to the escape of the fluid composition contained in the carpule as to the penetration of foreign bodies and microorganisms provided that said lips remain free of any trace of liquid composition in line with these lips.

The mechanical filling is therefore very challenging and remains a problem to be solved.

In addition, such carpules are produced in large numbers, and the method as well as the device must be adapted to be put into service on high-capacity and high-speed industrial production lines. This invention also proposes a sealing line that is suited for an application that is particular to carpules for needle-free injection.

The reproducibility is absolutely essential so as to impart the same parameters to all carpules produced and to result in a usage with consistent performances of the injection means that use said carpules.

It is also necessary to propose a method and introduction means that are of an acceptable cost.

### BRIEF DESCRIPTION OF THE DRAWINGS

The method and the insertion means according to this invention are now described in detail according to a particular, nonlimiting embodiment, this description being established for a carpule application without in any way being limiting to this type of container. This description relies on the set of accompanying drawings, drawings in which the different figures show, solely by way of illustration:

FIG. 1: a view in perspective of a carpule with its upper, distal first plug before being put into place, and its lower, proximal second plug in place,

FIG. 2: a diagrammatic view of the insertion means of the first plug,

FIGS. 3A to 3F: a view of a block diagram of the sealing method according to this invention, with the different steps,

FIG. 4: a view of a variant of the method with an additional step,

FIGS. 5A to 5B: a view of variant embodiments of the rod of the insertion means,

FIGS. 6A to 6H: a production line with the associated steps for the sealing using the device according to this invention.

### DESCRIPTION OF THE PREFERRED EMBODIMENTS

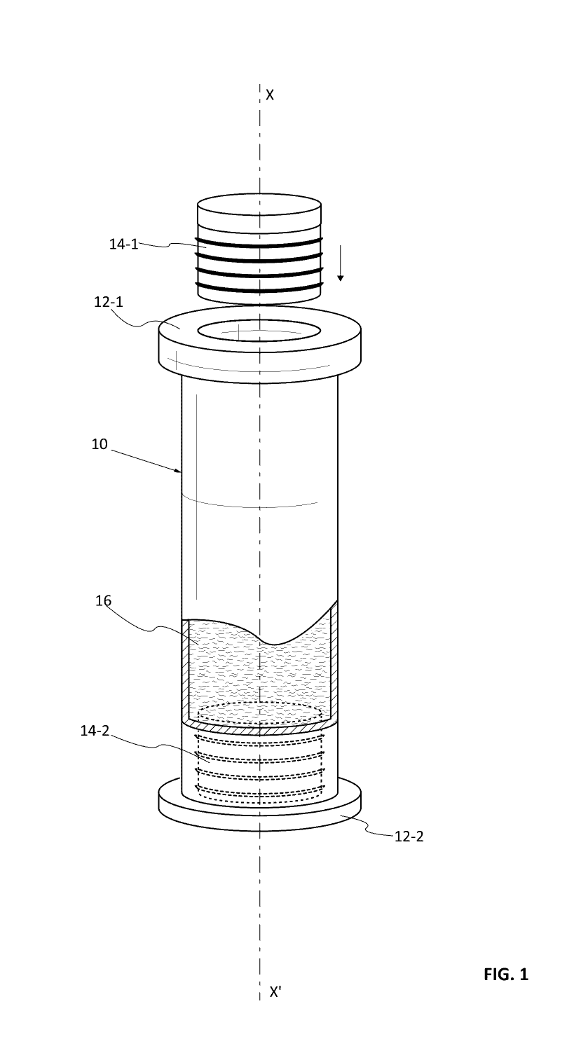

In FIG. 1, a carpule of known type has been shown, serving as a nonlimiting illustration in the following description. This cylindrical-shaped carpule comprises a tubular body 10, with two ends: an upper end 10-1 and a lower end 10-2.

This carpule comprises two crowns, upper 12-1 and lower 12-2. These crowns 12 are positioned on the outer periphery of the upper or distal and lower or proximal ends of the body 10.

Each crown is positioned in a plane perpendicular to the longitudinal axis XX′ of the carpule, passing through the longitudinal axis of the body 10.

The body and the crowns form a single-piece assembly and are generally made of glass, said glass being a thick glass so as to be able to withstand a high pressure.

The carpule in question is made to receive two plugs, made of a material of the elastomer type: an upper first plug 14-1 and a lower, releasing second plug 14-2.

The lower second plug 14-2 is introduced in a first operation so as to seal the body 10 in line with its second end 10-2, this second plug being a plug that releases the product under the action of the very high pressure.

The body 10 and the lower second plug 14-2 in place thus form a container designed to receive a liquid composition 16. The filling is performed in a known way, for example by means of a dispensing nozzle, not shown.

The upper first plug 14-1 must in its turn be introduced into the body 10 so as to seal the upper end of the tubular body 10.

The sealing method according to this invention proposes the following steps to achieve sealing with the first plug 14-1 by moving said plug to the immediate proximity of the free surface S for filling that is the free surface of the liquid composition 16.

The volume between this free surface S for filling and the plug is referred to as “head space” and must be minimized to solve the problems mentioned in the preamble.

The method consists in:
Introducing at least a portion of insertion means 18 parallel to the interior of the body 10 of the container, in this case the carpule,
Mechanically inserting the first plug 14-1 into the body 10, up to an insertion level of said plug in the body such that the air of the head space is evacuated between the plug and the insertion means 18, and
Withdrawing at least the portion of the insertion means 18 while leaving the first plug in place at the insertion level of the preceding step.

The insertion means 18 according to this invention comprise in particular a rod 18-1, with a circular cross-section in the simplified embodiment that is shown.

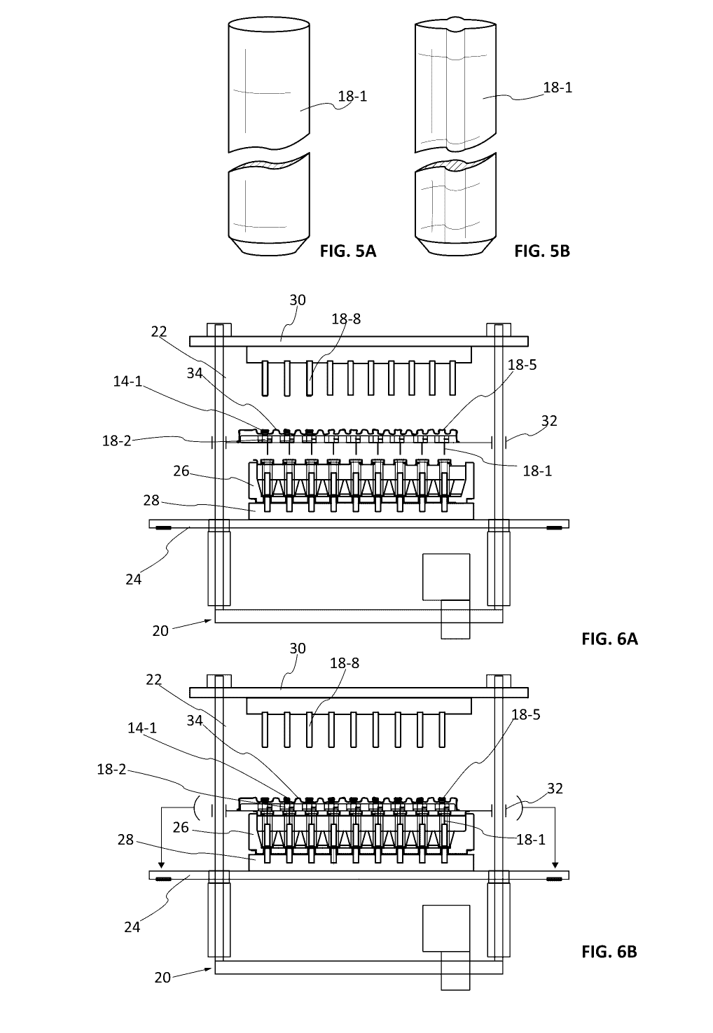

This rod 18-1 can assume different, irregular cross-sections, as shown in FIGS. 5A and 5B, particularly an elliptical cross-section and an elliptical cross-section with diametric lobes.

These cross-sections facilitate even more the evacuation of air by generating output channels C of greater cross-sections or by generating, for the same cross-section of channels, a greater mechanical strength of the rod, as explained previously.

When the first plug 14-1 is mechanically introduced, at least one generatrix of said plug, since it is cylindrical, rests against the rod 18-1 constituting a portion of the insertion means 18. This, therefore, generates at least one channel C for evacuation of air according to the translation of the plug in the body 10, along the axis XX′.

The rod 18-1 is then withdrawn from the body 10 of the carpule. This is made possible because the contact surface between the rod 18-1 and the upper first plug 14-1 generates friction forces that are less than those that are generated between the periphery of the plug and the inside wall of the body 10 of the carpule.

The first plug 14-1, made of elastomer material, resumes its initial shape and instantaneously compensates for the withdrawal of the rod 18-1.

Preferably, according to a characteristic of the method, the rod 18-1 is positioned with its lower end in immediate proximity of the free surface S for filling, while avoiding any contact with the liquid composition 16. Actually, such contact would have the consequence of polluting the space between the inside wall of the body 10 and the upper first plug 14-1, during the withdrawal of said rod 18-1.

According to another characteristic of this method, it is possible to add an additional step that consists in tilting the body 10 during its insertion. This tilting is performed along a generatrix, opposite the generatrix receiving the rod, so that the head space is concentrated on the side of the rod.

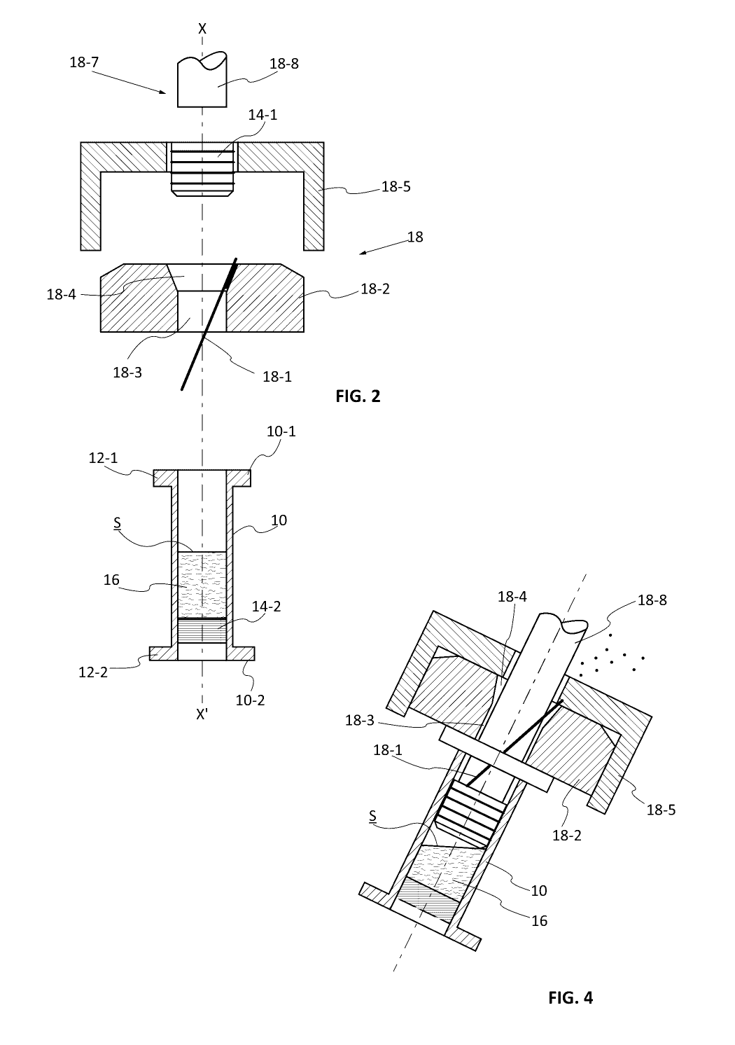

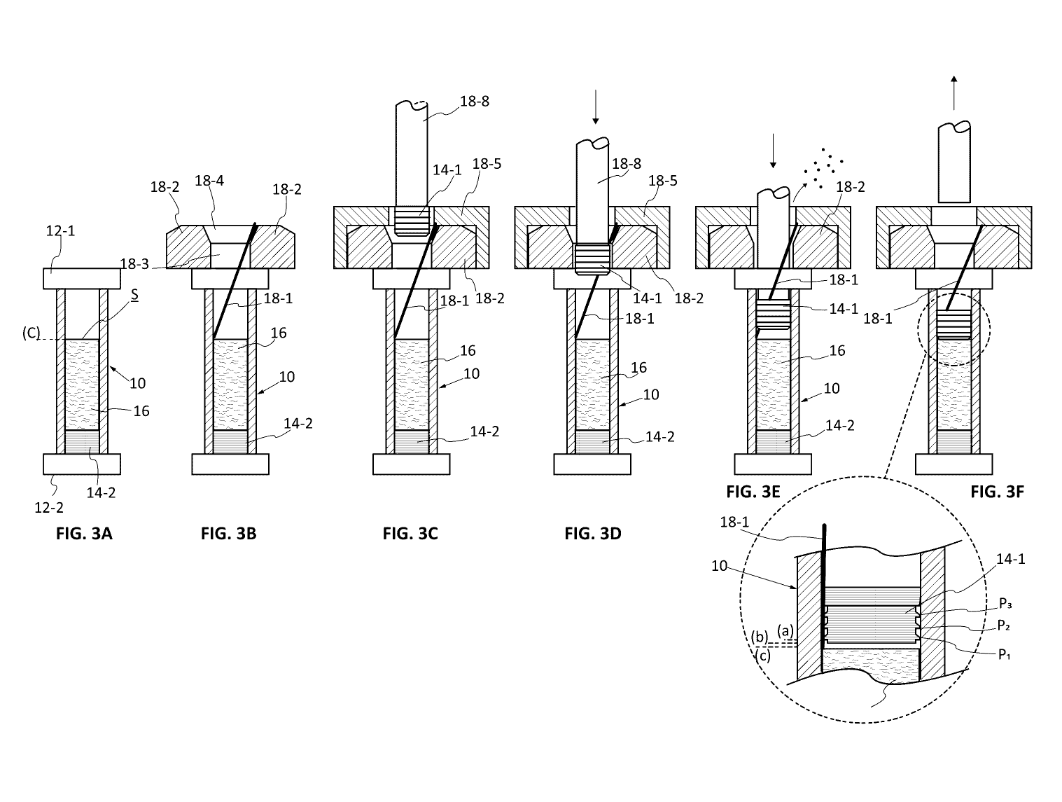

Means 18 for introducing the upper first plug 14-1 are now described in detail with regard to FIGS. 2 and 3, according to a preferred embodiment.

These figures also illustrate the complete device according to the invention, making it possible to implement the method that has just been described.

In FIG. 2, the introduction means 18 comprise a rod 18-1 integral with a guiding head 18-2 that comprises a passage 18-3 provided with a funnel-shaped entrance 18-4.

The guiding head 18-2 has a cylindrical and peripherally bevelled shape, designed to rest against and to use as a reference on the upper end 10-1, and more particularly on the upper crown 12-1.

The introduction means 18 also comprise a plug holder 18-5 designed to receive and to hold the upper first plug 14-1 in a pass-through housing 18-6 made in the center of said plug holder, and coaxially to the axis XX′.

The plug holder 18-5 has a profile combined with that of the guiding head 18-2 so as to cap said head and to ensure a coaxial positioning before introduction.

A mechanical actuator 18-7 is diagrammatically shown in the form of a plunger 18-8.

The implementation of the insertion means 18 that have just been described is now explained with regard to FIGS. 3 that show the corresponding summary diagram.

In FIG. 3A, a carpule has been shown with a body 10 and two crowns, upper 12-1 and lower 12-2. These crowns 12 are positioned on the outer periphery of the upper 10-1 and lower 10-2 ends of the body 10.

The lower second plug 14-2 is in place and seals the lower end 10-2.

The body 10 has received a volume of a liquid composition 16 whose free surface S for filling has a height (c).

The guiding head 18-2 is positioned against and referenced on the upper end 10-1 and against the crown 12-1.

In this position, the rod 18-1 comes immediately above the free surface S for filling, without contact, see FIG. 3B.

The guiding head 18-2 can also be capping on the crown for a better positioning.

In FIG. 3C, the plug holder 18-5 is returned coaxially onto the guiding head 18-2.

The passage 18-3 provided with a funnel-shaped entrance 18-4 is perfectly centered on the axis XX′.

The upper first plug 14-1 is held in the pass-through housing 18-6 made in the center of said plug holder 18-5, and is also found positioned coaxially relative to the axis XX′.

The plunger 18-8 is above the plug holder 18-5, standing by and also coaxial with the axis XX′.

In FIG. 3D, the plunger 18-8 is moved in translation according to the indication of the arrow of this figure.

The plunger 18-8 rests against the upper face of the upper first plug 14-1 and forces its introduction into the funnel-shaped entrance 18-4, which also centers said plug and causes a certain radial compression of said plug.

During the translation, the peripheral edge of the plug enters into contact with the rod 18-1, which generates at least one channel C that allows the evacuation of air, according to the insertion by translation of the upper first plug 14-1 into the body 10. The plug leaves the plug holder 18-5 that then no longer has a function.

In FIG. 3E, it is found that the first plug 14-1 descends and that the air escapes. The plunger has pushed the plug 14-1 to a referenced position (b) while leaving a very small head space.

The plug 14-1 generally consists of elastomer material and comprises in a fluidtight manner at least two lips, in this case three lips I1, I2, I3, with I1 being the closest to the lower end, followed by the upper part of the body of the plug. The dimensions are adapted as a function of needs, of the nature of the materials, the inside of the body 10 being able to undergo in advance a suitable surface treatment.

The liquid composition 16 has its free surface S for filling in a position c and the first lip I1 is in a position (a).

It is found that the position of the rod must be situated, at its lowest, immediately above the free surface S for filling of the liquid contained in the body of the container, at least above the position (c).

This prevents the liquid composition 16 from coming into contact with the end of the rod 18-1 and polluting it.

The first lip I1 therefore clearly ensures an effective first barrier, without a trace of liquid composition 16 invading between said lip and the inside wall of the body 10.

In this FIG. 3F, the plunger 18-8 is withdrawn, and the plug 14-1 remains in place. It is noted that the other lips I2 and I3 also resume their fluidtightness position because of the elastomeric nature of said material upon the withdrawal of the rod.

Also, the friction on this elastomer material remains completely negligible, and it does not cause any movement of the plug during the withdrawal of the rod.

Concerning the rod, so as not to damage in any way the inside face of the tubular body, to facilitate the installation of the first plug, it is possible to provide a surface treatment of said wall.

An advantageous solution also consists in making said rod from a material known under the name of PEEK that is polyether ketone, which exhibits mechanical properties that are largely sufficient for the application of this invention and whose friction coefficient is very limited.

It is enough then to withdraw the plug holder 18-5 that is plug-free, and then the guiding head 18-2.

These two withdrawals can be performed simultaneously.

According to an improvement of this invention, it is designed to be able to incline the body 10 slightly as shown in FIG. 4 so as to concentrate the volume of air at the end of pushing on the plug in a place that is situated in line with the end of the rod 14-1.

Thus, the head space is concentrated at one point.

This step is optional because, as a function of the products, the wettability of the liquid composition 16 is enough to come immediately under the lower face of the first plug 14-1. The head volume is then reduced to the peripheral crown situated under the first lip I1, which is extremely limited and completely acceptable particularly for the application considered of administration from a carpule under very high pressure.

This is all the more acceptable for single-dose syringes that are pre-filled with manual pressure.

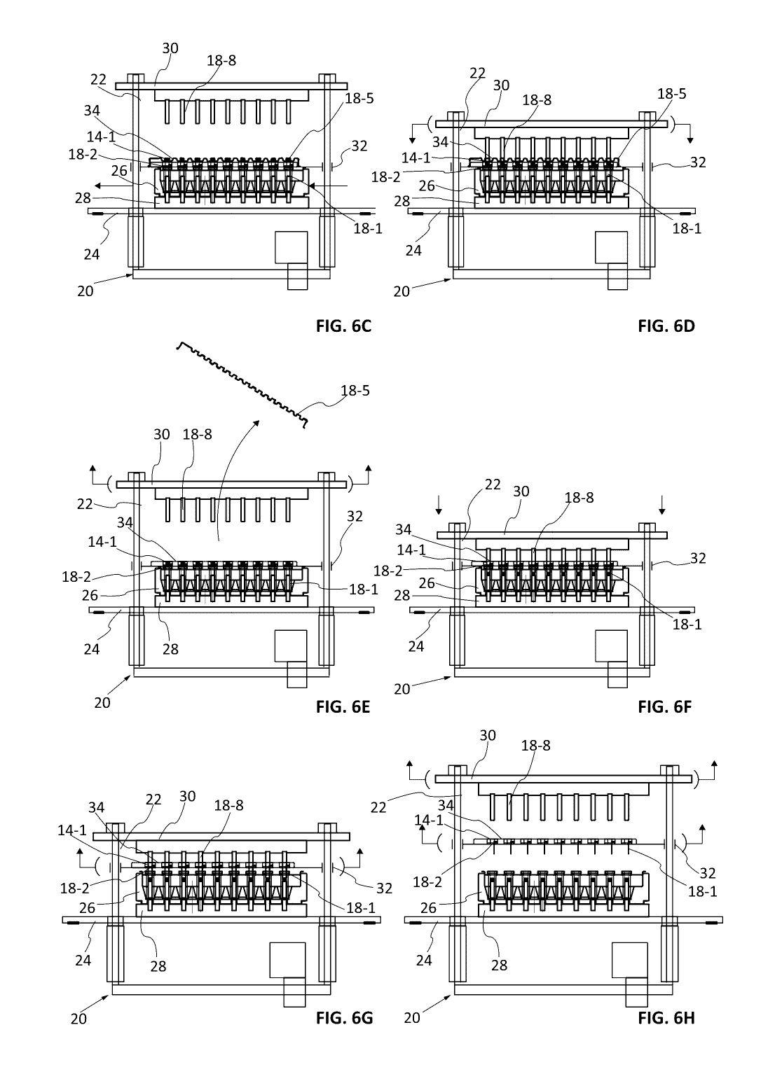

Concerning the method and the sealing means that make possible its implementation, it is found that it is possible to industrialize this method, and an illustration of an industrial production line is now described with regard to FIGS. 6.

The references in the industrial production line remain identical.

In this case, the line comprises 72 simultaneous positions.

In FIG. 6A, shown diagrammatically is a loading press that comprises a frame 20 with guiding columns 22, a lower plate 24 designed to receive a container-carrying tray 26, in this case carpules if the description is followed for this particular application to carpules. This lower plate 24 comprises means 28 making it possible to move the carpule-carrying tray 26 in horizontal translation.

An upper plate 30 that can move in translation on the guiding columns 22 is also provided. This upper plate 30 carries an assembly of plungers 18-1 with the same geometric distribution as that of the carpules.

An intermediate third plate 32 can also move in translation on the guiding columns 22, parallel to the upper plate 30. This intermediate plate 32 comprises a tray 34 with as many plug holders 18-2 and rods 18-1 as carpules with the same geometric distribution. The rods 18-1 are off-center relative to the plugs 14-1 so as to be positioned approximately in line with the periphery of each plug.

The frame 20 can also, although this is not shown, comprise a “dirty” zone and a “clean,” and even sterile, zone, so as to keep the carpules in the clean zone.

The frame 20 being in the configuration of FIG. 6A, the lower plate 24 carries a carpule-carrying tray 26, said carpules being filled with a liquid composition 16 and having received in advance a second plug for sealing the lower end 10-2.

These steps are not part of the invention because they are perfectly well known by themselves.

The intermediate plate 32 is positioned above the lower plate 24 to make possible the depositing of the carpule-carrying tray 26. The rods 18-1 are then centered in relation to the axis XX′ of the carpules, and the plugs 18-2 in the plug carrier are off-center.

In FIG. 6B, the first step is the descent of the intermediate plate 32 that ensures a placing of the rods 18-1 in a centered and therefore coaxial way with the axis XX′ of the carpules.

The step of FIG. 6C consists in causing a movement in horizontal translation until the rods come along an interior generatrix of the body 10 of each carpule.

The plugs 14-1 are then centered in relation to the carpules and the guiding heads 18-2 as well. The rods 18-1 are, themselves, off-center, positioned along an interior generatrix of the body 10.

This step is optional but nevertheless constitutes the best embodiment because it avoids the accidental resting of at least one rod 18-1 on the upper annular part 12-1 of a carpule, which could cause the breaking and at the very least the deformation of the rod in question.

In step 6D, the upper plate 30 is moved in translation downward, which causes the simultaneous descent of the plungers 18-8.

The plugs 14-1 are therefore withdrawn from the plug-carrying tray 34 and penetrate through each respective guiding head 18-2, in the funnel-shaped passage 18-4, and then in the passage 18-3.

The upper plate 30 with its plungers is then brought back into the initial position by a translation upward, as shown in FIG. 6E, to make possible the withdrawal of the plug-carrying tray 34.

In FIG. 6F, the upper plate 30 has descended again but lower to push the plugs 14-1 out of the guiding head, into the body 10, and to reduce the head space. Each plug is moved in translation from the guiding head that was holding it into the body of the corresponding carpule.

Since each plug is pushed in translation along the inside wall of the body 10 of each carpule and along the associated rod 18-1, the air or the neutral gas in the case of sealing under a neutral atmosphere can escape through the channels C thus formed, as explained above in the description.

The level of descent is regulated as a function of the filling data to obtain, for each carpule, a filling of the liquid composition to a level (c), a lower end of the plug 14-1 to a level (b), and an end of the rod to a level (a).

It is well understood that it is possible to provide the introduction of several rods for each carpule so as to generate more channels for evacuation of air or gas and/or to coordinate the sealing operation. This can make possible a better balancing of the plug during the descent.

FIG. 6G provides for the movement in translation of the intermediate plate 32 while leaving in place the plungers 18-8 and therefore the upper plate 30, in a low position. This avoids any movement of each plug during the withdrawal of the needles. The risk is slight but the hold of the plugs during the withdrawal of the needles is an additional guarantee.

The length of the plungers must be specified as a consequence.

FIG. 6H shows the return of the plate 30 to a high position.

The tray of full and sealed carpules with a head space, minimized, is ready to be prepared for marketing. The carpules of the tray can be incorporated into an applicator, particularly into casings of needle-free injection devices.

According to a variant of the method, the rod 18-1 and the plug 14-1 can be introduced simultaneously.

The sealing method thus makes it possible to minimize the head space, the sealing means are simple and industrial, and the production line meets industrial needs.

1. Method of sealing a container with a liquid, said container comprising a body (10) with an opening at at least one end and comprising at least one sealing plug (14-1), designed to seal said opening after filling, said filling defining a free surface S for filling, the filling having allowed a head space to remain in said container, the method comprising:
a) introducing at least one rod (18-1) parallel to an inside wall of the body (10) of said container,
b) mechanically inserting said plug (14-1) into the body (10), along the at least one insertion rod (18-1), generating at least one channel C, the air of the head space being evacuated between said plug and said rod,
c) withdrawing said at least one rod (18-1) while leaving said plug in place at the level of insertion of the preceding step.

2. Method of sealing a container with a body comprising an opening at at least one end, according to claim 1, wherein the steps a) for introduction of the rod (18-1) and b) for insertion of the plug (14-1) are simultaneous.

3. Method of sealing a container with a body comprising an opening at at least one end, according to claim 1, wherein the insertion rod (18-1) is introduced until an end of the insertion rod is immediately above the free surface S for filling of the liquid composition (16), contained in the body (10) of the container.

4. Method of sealing a container with a body comprising an opening at at least one end, according to claim 1, wherein, in the case of a plug (14-1) comprising at least one lip (I1-I3), the insertion rod (18-1) is introduced so that an end of the insertion rod is positioned in line with the lip (I1) closest to the free surface S of the liquid composition (16).

5. Method of sealing a container with a body comprising an opening at at least one end, according to claim 1, wherein, in the case of a cylindrical body (10), the direction of introduction of the rod (18-1) is parallel to a generatrix.

6. Method of sealing a container with a body comprising an opening at at least one end, according to claim 1, wherein the body (10) and/or the rod (18-1) and/or the plug (14-1) undergo a prior surface treatment or are made of a material having a low friction coefficient, to reduce the friction coefficient.

7. Method of sealing a container with a body comprising an opening at at least one end, according to claim 1, wherein the plug (14-1) is held in place mechanically during step c/for withdrawal of the rod (18-1).

8. Method of sealing a container with a body comprising an opening at at least one end, according to claim 1, wherein the body (10) of the container is tilted during the evacuation of the air by positioning the container so that the end of the rod (18-1) comes to a high point of the head space.

9. Method of sealing a container with a body comprising an opening at at least one end, according to claim 1, wherein the body (10) is a carpule comprising, in addition to the upper end (10-1) sealed by the sealing plug (14-1), another end (10-2) closed in advance with a releasing plug (14-2).

10. Device for sealing a container with a body comprising an opening at at least one end, for the implementing of the method according to claim 1, the device comprising a head (18-2) for guiding a plug (14-1) into said filled container, at least one rod (18-1) and a plug holder (18-5) as well as a mechanical actuator (18-7) comprising at least one plunger (18-8).

11. Device for sealing a container with a body comprising an opening at at least one end, according to claim 10, wherein the rod (18-1) has a disk-shaped cross-section.

12. Device for sealing a container with a body comprising an opening at at least one end, according to claim 10, wherein the rod (18-1) has an irregular cross-section.

13. Line for production of containers by implementing the method according to claim 1 and using a device comprising a head (18-2) for guiding a plug (14-1) into said filled container, at least one rod (18-1) and a plug holder (18-5) as well as a mechanical actuator (18-7) comprising at least one plunger (18-8), particularly for a use in needle-free syringes, each container comprising a body (10) with a first and a second open end (10-1, 10-2), wherein the device comprises a loading press comprising:
a frame (20) with guiding columns (22),
a lower plate (24) designed to receive a container-carrying tray (26), with a distribution geometry,
an upper plate (30), able to move in translation on the columns (22), provided with an assembly of plungers (18-1), with the same distribution geometry as the containers,
an intermediate plate (32) also able to move in translation on the guiding columns (22), parallel to said upper plate (30), provided with a tray (34) of plug holders (18-2) with as many plugs (14-1) and rods (18-1) as containers with the same geometric distribution.

14. Line for production of containers according to claim 13, wherein the lower plate (24) comprises means (28) making it possible to move the container-carrying tray (26) in horizontal translation.

15. Line for production of containers according to claim 13, wherein the rods (18-1) are off-center relative to the plugs (14-1) so as to be positioned approximately in line with the periphery of each plug (14-1).

16. Method of sealing a container with a body comprising an opening at at least one end, according to claim 2, wherein the insertion rod (18-1) is introduced until an end of the insertion rod is immediately above the free surface S for filling of the liquid composition (16), contained in the body (10) of the container.

17. Method of sealing a container with a body comprising an opening at at least one end, according to claim 2, wherein, in the case of a plug (14-1) comprising at least one lip (I1-I3), the insertion rod (18-1) is introduced so that an end of the insertion rod is positioned in line with the lip (I1) closest to the free surface S of the liquid composition (16).

18. Method of sealing a container with a body comprising an opening at at least one end, according to claim 3, wherein, in the case of a plug (14-1) comprising at least one lip (I1-I3), the insertion rod (18-1) is introduced so that an end of the insertion rod is positioned in line with the lip (I1) closest to the free surface S of the liquid composition (16).

19. Method of sealing a container with a body comprising an opening at at least one end, according to claim 2, wherein, in the case of a cylindrical body (10), the direction of introduction of the rod (18-1) is parallel to a generatrix.

20. Method of sealing a container with a body comprising an opening at at least one end, according to claim 3, wherein, in the case of a cylindrical body (10), the direction of introduction of the rod (18-1) is parallel to a generatrix.
